SEQUENCE LISTING (4 sequences)

SEQ ID NO: 1 - protein, 7 aa, Artificial Sequence
  TEV cleavage site
ENLYFQG

SEQ ID NO: 2 - protein, 7 aa, Artificial Sequence
  TEV cleavage site
ENLYFQS

SEQ ID NO: 3 - protein, 17 aa, Artificial Sequence
  tryptic peptide
CLKEASLTLLDEEDAKK

SEQ ID NO: 4 - protein, 132 aa, Artificial Sequence
  tryptic peptide
MKTGEQRVKIATLLSENEKKIVDKASQDLWRRRPDFIAPGGNAFGQRERALCLRDYGWYLRLITYCLLAGDKDPIESIGLIGVREMYNSLGVPVPGMVESIRCLKEASLTLLDEEDAKKTAPYFDYIIQAMS

(19) United States
(12) Patent Application Publication
(10) Pub. No.: US 2019/0025316 A1
(43) Pub. Date: Jan. 24, 2019

(54) PROTEIN INTERACTOR DETECTION SYSTEMS

(71) Applicant: THE REGENTS OF THE UNIVERSITY OF CALIFORNIA, Oakland, CA (US)

(72) Inventors: Xiaokun SHU, San Francisco, CA (US); William DEGRADO, San Francisco, CA (US); Tsz-Leung TO, San Francisco, CA (US); Hyunil JO, San Francisco, CA (US)

(21) Appl. No.: 16/069,476

(22) PCT Filed: Jan. 12, 2017
(86) PCT No.: PCT/US17/13243

§ 371 Date: Jul. 11, 2018

Related U.S. Application Data
(60) Provisional application No. 62/277,863, filed on Jan. 12, 2016.

(51) Int. Cl. G01N 33/58; G01N 33/68

(52) U.S. Cl. CPC G01N 33/582; G01N 33/6803

(57) ABSTRACT

Provided herein are systems, methods and reagents for determining interactors (proteins or nucleic acids) that interact with a protein of interest. The subject system, methods and reagents advantageously allow for the identification of weak and transient protein-protein interactions. Such subject system, methods and reagents are useful, for example, for the determination of specific protein-interactor interactions that exist in particular diseases. Determination of these differences is useful, for example, in the drug development for the treatment of such diseases.

### GOVERNMENT SUPPORT

This invention was made with government support under Grant No. GM105446 and GM054616 awarded by the National Institutes of Health. The government has certain rights in the invention.

### BACKGROUND

Genome sequences of all important model organisms are now available, owing to the technological advances in high throughput DNA sequencing. An important next step in understanding biology and disease is to identify protein interactome and to characterize protein machines, which carry out every major cellular process and maintain homeostasis of organism (Seebacher et al., Cell 144, 1000, 1000.e1. (2011); Bonetta, Nature 468, 851-854 (2010); Perkel, Science 329, 463-465 (2010); and Alberts, Cell 92, 291-294 (1998)). However, unlike the genome, a protein interactome is dynamic and protein machines may include weakly associated components, which hit the blind spots of current technologies.

Protein-protein interaction networks underlie most cellular processes. Protein machines execute most cell functions, such as the replisome for DNA replication; RNA polymerase complex for DNA transcription; ribosomes for protein synthesis; and anaphase-promoting complex that drives cell cycle progression in both early embryo development and adult homeostasis. To understand biology and disease and to identify new therapeutic targets, it is necessary to search, analyze and visualize protein-protein interaction networks. Indeed, over the past decade especially since completeness of the human genome project, much effort has been devoted to the study of protein interactome. Large databases have been generated, such as BioGRID and IntAct. Now researchers and funding agencies are gearing up to map the human interactome (including protein-protein and protein-DNA interactions). For example, the Canada Foundation for Innovation along with its partners awarded nearly US$20 million “to create a technology platform to map the human interactome.”

The previous technologies for mapping protein-protein interactions have blind spots. Affinity purification coupled to mass spectrometry (AP/MS) separates protein complexes from cell extracts, followed by characterization of the components based on mass. Some partners whose interactions depend on cellular environment are thus likely to be missed. While chemical crosslinking may be used, it lacks specificity. The second type of methods detects pair-wise protein interactions in a cellular context, including yeast two-hybrid assay (Y2H), protein complementation assay (PCA), luminescence-based mammalian interactome (LUMIER), and mammalian protein-protein interaction trap (MAPPIT). However, each method only detects a small percentage of the interactions (<50%) as demonstrated in a recent study.

Accordingly, there is a need for new systems, reagents, and methods for the determination of protein-protein interactions.

### SUMMARY

Provided herein are systems, methods and reagents for determining interactors (proteins or nucleic acids) that interact with a protein of interest. The subject system, methods and reagents advantageously allow for the identification of weak and transient protein-protein and protein-interactions. Such subject system, methods and reagents are useful, for example, for the determination of specific protein-interactor interactions that exist in particular diseases. Determination of these differences is useful, for example, in the drug development for the treatment of such diseases.

In a first aspect, provided herein is an interactor detection molecule according to the formula:

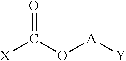

where: X is a label; A is an optional linker; and Y is a reactive group capable of reacting with a cysteine, lysine histidine or serine side chain upon oxidation by a singlet oxygen (¹O₂). In some embodiments, the label X is a radioisotope, a stable isotope, a fluorophore, an electron dense metal, biotin, a nucleic acid or an antibody epitope. In certain embodiments, the linker A is an alkyl chain, an aryl, a heteroaryl, or polyethylene glycol.

In some embodiments, the reactive group Y is a thiol, a furan, a pyrrole, an enol ether, a phenol or a naphthol or derivative. In certain embodiments, the thiol is selected from an alkyl thiol, an aryl thiol, a cysteine and a peptide that includes a cysteine. In certain embodiments, X is biotin, A is CH₂CH₂, and Y is a thiol containing group.

In another aspect, provided herein is an interactor detection molecule having a formula:

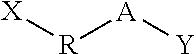

where X is a label; R is a cleavable peptide; A is an optional linker; and Y is a reactive group capable of reacting with a cysteine, lysine histidine or serine side chain upon oxidation by a singlet oxygen (¹O₂). In some embodiments, the label X is a radioisotope, a stable isotope, a fluorophore, an electron dense metal, biotin, a nucleic acid or an antibody epitope. In certain embodiments, the linker A is an alkyl chain, an aryl, a heteroaryl, or polyethyleneglycol. In some embodiments, the cleavable peptide R includes a protease cleavage site. In exemplary embodiments, the protease cleavage site is a Tobacco Etch Virus protease cleavage site. In certain embodiments, the linker A is an alkyl chain, an aryl, a heteroaryl, or polyethylene glycol. In some embodiments, X is biotin and Y is a cysteine.

In exemplary embodiments, the reactive group is a thiol, a furan, a pyrrole, an enol ether, a phenol or a naphthol or derivative. In some embodiments, the thiol is selected from an alkyl thiol, an aryl thiol, a cysteine and a peptide that includes a cysteine.

In another aspect, provided herein is a system for determining interactors that interact with a protein of interest comprising: In some embodiments, the SOG-POI protein includes a singlet oxygen photosensitizer linked to a protein of interest, where the singlet oxygen photosensitizer is capable of producing singlet oxygen (¹O₂) when illuminated with a light source; and any one of the interactor detection molecules provided herein.

In another aspect, provided herein is a cell comprising: a SOG-POI protein comprising a singlet oxygen photosensitizer linked to a protein of interest, where the singlet oxygen photosensitizer is capable of producing singlet oxygen (¹O₂) when illuminated with a light source; and any one of the interactor detection molecules described herein.

In another aspect, provided herein is a method for determining interactors that interact with a protein of interest. In some embodiments, the method includes the steps of: a) introducing into a cell a SOG-POI protein and an interactor detection molecule selected from any one of the interactor detection molecules described herein, where the SOG-POI protein includes a singlet oxygen photosensitizer linked to a protein of interest, and where the singlet oxygen photosensitizer is capable of producing singlet oxygen (¹O₂) when illuminated with a light source; b) illuminating the singlet oxygen photosensitizer with a light source, thereby producing singlet oxygen (¹O₂) that oxidizes the interactor detection molecule to form a reactive detection intermediate, where the reactive detection intermediate binds with a interaction protein that interacts with the protein of interest, thereby labeling the interactor; and c) characterizing the interactor. In some embodiments, the c) characterizing is carried out by isolating the interactive intermediate bound interactors and the interactor is sequenced by mass spectrometry. In some embodiments, X of the interactor detection molecule is a biotin and isolation of the interactive protein is performed using a substrate attached to streptavidin.

### BRIEF DESCRIPTION OF THE DRAWINGS

FIG. 1 provides a schematic of one embodiment of the subject systems and methods for detection and labeling of proteins that interact with a protein of interest (POI). (1) Upon illumination by a light source, a singlet oxygen photosensitizer (mimiSOG) linked to a protein of interest converts O₂ to singlet oxygen (¹O₂). In (2) and (3) a reactive group on a protein interaction detection molecule undergoes an oxidation reaction by the singlet oxygen to product a reactive intermediate. The reactive intermediate is able to then interact with side chains of proteins that interact with the protein of interest (3) (e.g., thiol in cysteine), thereby labeling proteins that interact with the protein of interest.

FIG. 2 provides another schematic showing one embodiments of the subject and systems provided herein. FIG. 2A: (1) Upon illumination by a light source, a singlet oxygen photosensitizer (mimiSOG) linked to a protein of interest converts O₂ to singlet oxygen (¹O₂). (2) the singlet oxygen converts a protein interaction detection molecule (white triangle) into a reactive intermediate (orange triangle). The reactive intermediate is able to then interact with side chains of proteins that interact with the protein of interest (3) (e.g., thiol in cysteine), thereby labeling interactors that interact with the protein of interest (e.g., proteins and nucleic acids) (4). In certain embodiments, the protein interaction detection molecule includes a label (e.g., biotin) that allows for separation of proteins that interact with the protein of interest. For example, separation can be performed using a streptavidin/biotin system (5). The interaction protein can be subsequently characterized by any suitable technique, for example, mass spectrometry techniques (6). FIG. 2B shows a singlet oxygen photosensitizer (SOG) linked to a protein of interest (POI) and an interaction protein (X) bound to the protein of interest. Upon illumination by a light source, the SOG converts O₂ to singlet oxygen. The singlet oxygen converts protein interaction detection molecules (white triangles) into a reactive intermediate (orange triangles) that are able to label the interaction protein (X).

FIG. 3 shows a proof in concept model for an embodiments of the systems and methods provided herein using an exemplary interaction protein detection molecule (biotin-conjugated thiol-containing molecule: 2-mercaptoethyl 5-((3aS,4S,6aR)-2-oxohexahydro-1H-thieno[3,4-d]imidazol-4 yl)pentanoate, with chemical formula: C₁₂H₂₀N₂O₃S₂). A singlet oxygen photosensitizer (SOG) was fused to a protein of interest, skp2, an F-box protein of SCF (Skp1-cullin-F-Box) ubiquitin ligase. SCF is a multi-protein complex, composed of four subunits: Skp1 (S-phase kinase-associated protein 1), cullin1, a RING finger protein (RBX1/HRT1/ROC1) and a variable F-box protein. The SOG-skp2 fusion protein was expressed in cultured mammalian cells, HeLa. The interaction protein detection molecules were added to the cells, followed by illumination with 480/30 nm light for 10-30 minutes. The cells were lysed, followed by centrifugation. The labeled interaction proteins were incubated with trypsin and loaded to streptavidin beads, and separated from the rest by streptavidin-biotin interaction. The labeled interaction peptides were eluted by incubation with amine hydroxyl, which cleaved the labeled peptides from streptavidin beads. These interaction peptides were identified by mass spectrometry.

FIG. 4 is a demonstration of the photo-activatable interaction protein labeling as described herein. Purified singlet oxygen photosensitizer (mimiSOG) APC fusion protein (1 μM) reacted with various concentrations of biotin-thiol under blue light illumination, followed by streptavidin purification and elution with desthiobiotin.

FIG. 5 provides a mass spectrometric analysis of the protein labeling by biotin-thiol. (a). Primary sequence of allophycocyanin (APC) indicates three cysteines (highlighted in red). The tryptic peptide containing Cys103 is highlighted in blue. (b). Modeled structure of APC (Swiss-Model) suggests that Cys103 resides on the surface, whereas Cys52 and Cys66 are buried inside the protein. The conserved Cys52 is likely linked to phycocyanobilin (in purple). (c). Mass spectrum of the identified tryptic peptide containing Cys103 conjugated to the biotin-thiol, as well as various fragment ions derived from this peptide. (d) Nomenclature of the fragment ions.

### DETAILED DESCRIPTION

### Definitions

Unless specifically indicated otherwise, all technical and scientific terms used herein have the same meaning as commonly understood by those of ordinary skill in the art to which this invention belongs. In addition, method or materials that are substantially equivalent to a method or material described herein can be used in the practice of the present invention. For purposes of the present invention, the following terms are defined.

The term “isolated” as used herein with respect to nucleic acids, such as DNA or RNA, refers to molecules separated from other DNAs, or RNAs, respectively that are present in the natural source of the macromolecule. Isolated is meant to include nucleic acid fragments which are not naturally occurring as fragments and would not be found in the natural state. The term isolated as used herein also refers to a nucleic acid or peptide that is substantially free of cellular material, viral material, or culture medium when produced by recombinant DNA techniques, or chemical precursors, or other chemicals when chemically synthesized.

More broadly, the term “isolated” or “purified” refers to a material that is substantially or essentially free from other components that normally accompany the material in its native state in nature. Purity or homogeneity generally are determined using analytical chemistry techniques such as polyacrylamide gel electrophoresis, high-performance liquid chromatography (HPLC), and the like. A polynucleotide or a polypeptide is considered to be isolated when it is the predominant species present in a preparation. Generally, an isolated protein or nucleic acid molecule represents greater than 50% of the macromolecular species present in a preparation, usually represents greater than 80% or 90% of all macromolecular species present, often represents greater than 95%, of the macromolecular species, and, in particular, may be a polypeptide or polynucleotide that purified to essential homogeneity such that it is the only species detected when it is examined using conventional methods for determining the purity of such a molecule.

The term “naturally occurring” is used to refer to a protein, nucleic acid molecule, cell, or other material that exists in the natural world, for example, a polypeptide or polynucleotide sequence that is present in an organism, including in a virus. In general, at least one instance of a naturally occurring material existed in the world prior to its creation, duplication, or identification by a human. A naturally occurring material can be in its form as it exists in the natural world, or can be modified by the hand of man such that, for example, it is in an isolated form.

The term “label” refers to a composition that is detectable with or without instrumentation, for example, by visual inspection, spectroscopy, or a photochemical, biochemical, immunochemical, or chemical reaction. Useful labels include, for example, phosphorus-32, a fluorescent dye, a fluorescent protein, an electron-dense reagent, an enzyme such as is commonly used in an ELISA, or a small molecule (such as biotin, digoxigenin, or other haptens or peptides) for which an antiserum or antibody, which can be a monoclonal antibody, is available. It will be recognized that a fluorescent protein variant of the invention, which is itself a detectable protein, can nevertheless be labeled so as to be detectable by a means other than its own fluorescence, for example, by incorporating a radionuclide label or a peptide tag into the protein so as to facilitate, for example, identification of the protein during its expression and the isolation of the expressed protein, respectively. A label useful for purposes of the present invention generally generates a measurable signal such as a radioactive signal, fluorescent light, enzyme activity, and the like, either of which can be used, for example, to quantitate the amount of the fluorescent protein variant in a sample.

The term “polypeptide” or “protein” refers to a polymer of two or more amino acid residues. The terms apply to amino acid polymers in which one or more amino acid residue is an artificial, chemical analogue of a corresponding naturally occurring amino acid, as well as to polymers of naturally occurring amino acids. The term “recombinant protein” refers to a protein that is produced by expression of a nucleotide sequence encoding the amino acid sequence of the protein from a recombinant DNA molecule.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters. A comparison window includes reference to a segment of any one of the number of contiguous positions selected from the group consisting of from 20 to 600, usually about 50 to about 200, more usually about 100 to about 150 in which a sequence can be compared to a reference sequence of the same number of contiguous positions after the two sequences are optimally aligned. Methods of alignment of sequences for comparison are well-known in the art. Optimal alignment of sequences for comparison can be conducted (e.g., by the local homology algorithm of Smith & Waterman, Adv. AppL Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection, e.g., Current Protocols in Molecular Biology (Ausubel et al., eds. 1995 supplement)).

Furthermore, it will be recognized that individual substitutions, deletions or additions that alter, add or delete a single amino acid or a small percentage of amino acids (typically less than 5%, and generally less than 1%) in an encoded sequence can be considered conservatively modified variations, provided alteration results in the substitution of an amino acid with a chemically similar amino acid. Conservative amino acid substitutions providing functionally similar amino acids are well known in the art, including the following six groups, each of which contains amino acids that are considered conservative substitutes for each another:

1) Alanine (Ala, A), Serine (Ser, S), Threonine (Thr, T);

2) Aspartic acid (Asp, D), Glutamic acid (Glu, E);

3) Asparagine (Asn, N), Glutamine (Gln, Q);

4) Arginine (Arg, R), Lysine (Lys, K)

5) Isoleucine (Ile, I), Leucine (Leu, L), Methionine (Met, M), Valine (Val, V); and

6) Phenylalanine (Phe, F), Tyrosine (Tyr, Y), Tryptophan (Trp, W).

Two or more amino acid sequences or two or more nucleotide sequences are considered to be “substantially identical” or “substantially similar” if the amino acid sequences or the nucleotide sequences share at least 90% sequence identity with each other, or with a reference sequence over a given comparison window. Thus, substantially similar sequences include those having, for example, at least 90% sequence identity, at least 95% sequence identity, at least 97% sequence identity, or at least 99% sequence identity.

The term “alkyl,” by itself or as part of another substituent, means, unless otherwise stated, a straight or branched chain, or cyclic hydrocarbon radical, or combination thereof, which may be fully saturated, mono- or polyunsaturated and can include di- and multivalent radicals, having the number of carbon atoms designated (i.e. C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkyl,” unless otherwise noted, is also meant to include those derivatives of alkyl defined in more detail below, such as “heteroalkyl.” Alkyl groups, which are limited to hydrocarbon groups are termed “homoalkyl”.

The term “alkylene” by itself or as part of another substituent means a divalent radical derived from an alkane, as exemplified, but not limited, by —CH₂CH₂CH₂CH₂—, and further includes those groups described below as “heteroalkylene.” Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms.

The terms “alkoxy,” “alkylamino” and “alkylthio” (or thioalkoxy) are used in their conventional sense, and refer to those alkyl groups attached to the remainder of the molecule via an oxygen atom, an amino group, or a sulfur atom, respectively.

The term “heteroalkyl,” by itself or in combination with another term, means, unless otherwise stated, a stable straight or branched chain, or cyclic hydrocarbon radical, or combinations thereof, consisting of the stated number of carbon atoms and at least one heteroatom selected from the group consisting of O, N, Si and S, and wherein the nitrogen and sulfur atoms may optionally be oxidized and the nitrogen heteroatom may optionally be quaternized. The heteroatom(s) O, N and S and Si may be placed at any interior position of the heteroalkyl group or at the position at which the alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. Up to two heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

The term “aryl” means, unless otherwise stated, a polyunsaturated, aromatic, hydrocarbon substituent which can be a single ring or multiple rings (preferably from 1 to 3 rings) which are fused together or linked covalently. The term “heteroaryl” refers to aryl groups (or rings) that contain from one to four heteroatoms selected from N, O, and S, wherein the nitrogen and sulfur atoms are optionally oxidized, and the nitrogen atom(s) are optionally quaternized. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

Each of the above terms (e.g., “alkyl,” “heteroalkyl,” “aryl” and “heteroaryl”) include both substituted and unsubstituted forms of the indicated radical. Preferred substituents for each type of radical are provided below.

Substituents for the alkyl, and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) are generally referred to as “alkyl substituents” and “heteroakyl substituents,” respectively, and they can be one or more of a variety of groups selected from, but not limited to: —OR′, ═O, ═NR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂ in a number ranging from zero to (2m′+1), where m′ is the total number of carbon atoms in such radical. R′, R″, R′″ and R″″ each preferably independently refer to hydrogen, substituted or unsubstituted heteroalkyl, substituted or unsubstituted aryl, e.g., aryl substituted with 1-3 halogens, substituted or unsubstituted alkyl, alkoxy or thioalkoxy groups, or arylalkyl groups. When a compound of the invention includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, the aryl substituents and heteroaryl substituents are generally referred to as “aryl substituents” and “heteroaryl substituents,” respectively and are varied and selected from, for example: halogen, —OR′, ═O, ═NR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″)═NR′″, —S(O)R′, —S(O)₂R′, —S(O)₂NR′R″, —NRSO₂R′, —CN and —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″ and R″″ are preferably independently selected from hydrogen, (C₁-C₈)alkyl and heteroalkyl, unsubstituted aryl and heteroaryl, (unsubstituted aryl)-(C₁-C₄)alkyl, and (unsubstituted aryl)oxy-(C₁-C₄)alkyl. When a compound of the invention includes more than one R group, for example, each of the R groups is independently selected as are each R′, R″, R′″ and R″″ groups when more than one of these groups is present.

Two of the aryl substituents on adjacent atoms of the aryl or heteroaryl ring may optionally be replaced with a substituent of the formula -T-C(O)—(CRR′)_{q}—U—, wherein T and U are independently —NR—, —O—, —CRR′— or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′— or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X—(CR″R′″)_{d}—, where s and d are independently integers of from 0 to 3, and X is —O—, —NR′—, —S—, —S(O)—, —S(O)₂—, or —S(O)₂NR′—. The substituents R, R′, R″ and R′″ are preferably independently selected from hydrogen or substituted or unsubstituted (C₁-C₆)alkyl.

In one aspect, provided herein is a system for labeling interactors (e.g., proteins and nucleic acids) that interact with a protein of interest. As used herein, an “interactor” refers to a protein or nucleic acid that binds to a protein of interest (POI). In some embodiments, the system includes (1) a singlet oxygen generating (SOG) sensitizer (e.g. a photosensitizer) linked to the protein of interest and; (2) an interactor detection molecule. In certain embodiments, the SOG, when illuminated with a light source, is capable of producing singlet oxygen (¹O₂) from O₂. Such singlet oxygen, in turn, converts the interactor detection molecule into a reactive intermediate. The reactive intermediate is then capable of labeling nearby interactors (e.g., proteins and nucleic acids) that interact with the protein of interest. Without being bound by any particular theory of operation, it is believed that specific labeling of local interactors (e.g., proteins and nucleic acids) is achieved due to the short diffusion distance of ¹O₂ in cells. Such a photoactivatable protein labeling system advantageously allows for the labeling and characterization of interactors (e.g., proteins and nucleic acids) that have been difficult to detect using previous approaches (e.g., affinity based approaches), in a spatially and temporally controlled manner. Features of the subject system are now discussed in detail below.

### SOG-POI Fusion Protein

In one aspect, provided herein is a singlet oxygen generating (SOG) sensitizer linked to a protein of interest (e.g., a SOG-POI fusion protein). Such SOG-POI fusion proteins are used in the subject systems provided herein for the labeling of interactors (e.g., proteins and nucleic acids) that interact with the protein of interest. SOG-POI fusion proteins can be made by any suitable method known in the art, including known, recombinant DNA, molecular cloning techniques and expression systems as described herein.

Any suitable singlet oxygen generating sensitizer may be included in the SOG-POI fusion protein. SOG sensitizers are capable of generating singlet oxygen upon activation by an activator. In some embodiment, the SOG sensitizer is a SOG photosensitizer. Useful photosensitizers include those that, upon illumination for a light source, are capable of using energy from the light source to generate reactive singlet oxygen. Exemplary photosensitizers include, but are not limited, to strongly light-absorbing organic dye molecules such as Rose Bengal, erythrosine, eosin, methylene blue, porphyrines and phthalocyanines. In some embodiments, the photosensitizer is capable of converting oxygen molecules to singlet oxygen upon excitation at a wavelength of from about 380 nm to about 1200 nm. Particular examples of ranges of light wavelength that may be utilized include a lower range limit of about 380 nm, about 400 nm, about 425 nm, about 450 nm, about 475 nm, about 500 nm, about 525 nm, about 550 nm, about 575 nm, about 600 nm, about 650 nm, about 675 nm, about 700 nm, about 725 nm, about 750 nm, about 775 nm, about 800 nm, about 825 nm, about 850 nm, and the like, and an upper range limit of about 1200 nm, about 1175 nm, about 1150 nm, about 1125 nm, about 1100 nm, about 1025 nm, about 1000 nm, about 975 nm, about 950 nm, about 925 nm, about 900 nm, about 875 nm, about 850 nm, about 825 nm, about 800 nm, about 775 nm, about 750 nm, and the like. In certain embodiments, the photosensitizer is capable of converting oxygen molecules to singlet oxygen upon excitation with visible violet light, indigo light, blue light, green light, yellow light, orange light or red light. In certain embodiments, the photosensitizer is capable of converting oxygen molecules to singlet oxygen upon excitation with blue light (450-490 nm). In some embodiments, the SOG photosensitizer does not require exogenous cofactors. Other photosensitizers that can be used in the subject SOG-POI fusion proteins include, but are not limited to, those described in U.S. Pat. Nos. 8,962,797; 8,881,741, and 8,748,446 and Shu et al., Biology 9, e1001041 (2011), each of which is incorporated herein by reference.

The protein of interest (POI) can be any suitable protein of interest. Proteins of interest include, but are not limited to, a receptor ligand protein, a signal transduction pathway protein, a a metabolic pathway protein or a fragment thereof. In some embodiments, the protein of interest is a protein fragment (e.g., a polypeptide). In some embodiments, the protein of interest is 1,000 amino acids or less, 950 amino acids or less, 900 amino acids or less, 850 amino acids or less, 800 amino acids or less, 750 amino acids or less, 700 amino acids or less, 650 amino acids or less, 600 amino acids or less, 550 amino acids or less, 500 amino acids or less, 450 amino acids or less, 400 amino acids or less, 350 amino acids or less, 300 amino acids or less, 250 amino acids or less, 200 amino acids or less, 150 amino acids or less, 100 amino acids or less, 95 amino acids or less, 90 amino acids or less, 85 amino acids or less, 80 amino acids or less, 75 amino acids or less, 70 amino acids or less, 65 amino acids or less, 60 amino acids or less, 55 amino acids or less, 50 amino acids or less, 45 amino acids or less, 40 amino acids or less, 35 amino acids or less, 30 amino acids or less, 25 amino acids or less, 20 amino acids or less, 15 amino acids or less or 10 amino acids or less.

In certain embodiments, proteins of interest include one or more structural domains that are known to participate in protein-protein interactions or protein-nucleic acid interactions. Structural domains that are known to participate in such interactions include, but are not limited to, Src homology 2 (SH2) domains, Src homology 3 (SH3) domains; phosphotyrosine-binding (PTB) domains, LIM domains; SAM domains; PDZ domains; FERM domains; calponin homology (CH) domains; pleckstrin homology domains; WW domains; and WS×WS motifs.

### Interactor Detection Molecules

In another aspect, provided herein is an interaction protein detection molecules. Such interaction protein detection molecules are useful, for example, in conjunction with the SOG-POI fusion proteins described above, as part of the subject interactor detection systems provided herein. Upon illumination, a SOG-POI fusion protein converts O₂ into singlet oxygen ¹O₂. Such singlet oxygen, in turn, is capable of converting an interactor detection molecule into a reactive intermediate that labels nearby POI interactors (e.g., proteins and nucleic acids). Without being bound by any particular theory of operation, it is believed that specific labeling of local interactors (e.g., proteins and nucleic acids) is achieved due to the short diffusion distance of ¹O₂ in cells (˜30 nm within half-life); singlet oxygen molecules produced by SOG-POI fusion proteins in turn convert nearby interaction protein detection molecules into reactive intermediates. Such reactive intermediates then label POI interactors (e.g., nucleic acids and proteins) that localize with the protein of interest. Interactor detection molecules can be made using any suitable method known in the art. In some embodiments, the subject interactor detection molecules are designed so that they are small enough to be cell permeable.

In some embodiments, the interactor detection molecule is according to the formula:

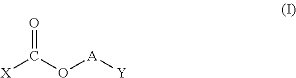

wherein: X is a label; A is an optional linker; and Y is a reactive group capable of interaction with an interactor (e.g., a protein or nucleic acid that interacts with the protein of interest) upon oxidation by a singlet oxygen (¹O₂). In an exemplary embodiment, X is biotin, A is CH₂CH₂, and Y is a thiol containing group.

The label X can be any label that is useful for the detection of the detection of interactors that interact with the protein of interest. In certain embodiments, the label also allows for the purification in addition to the detection of the interactor. Such purified interactors can are subsequently analyzed using suitable methods known in the art (e.g., mass spectrometry for proteins or DNA sequencing for nucleic acid interactors). Useful labels include, but are not limited to, radioisotopes (e.g., ³H, ³⁵S, ³²P, or ¹²⁵I), stable isotopes (e.g., C, N or O), fluorophores, electron dense metals, enzymes (e.g., alkaline phosphatase or horseradish peroxidase), biotins, digoxigenin, nucleic acids, antibody epitopes and any combination thereof. In some embodiments, any entity that is capable of binding to another entity may be used, including without limitation, epitopes for antibodies, ligands for receptors, and nucleic acids, which may interact with a second entity through means such as complementary base pair hybridization. In certain embodiments, the label X is a biotin label.

After the labeling reaction, any method that allows the detection of labeled polypeptides may be used to identify, isolate and analyze the labeled polypeptides. For example, the skilled artisan will recognize that interactors containing a biotin label can be isolated or detected using avidin-related proteins such as avidin itself, streptavidin, and neutravidin. Thus, neutravidin beads may be used to isolate biotin labeled polypeptides from complex mixtures or streptavidin linked to horseradish peroxidase may be used to identify biotin labeled polypeptides after protein separation by a procedure such as electrophoresis and avidin blotting.

Y can be any reactive group that is capable of attaching to an interactor (i.e., a protein or nucleic acid that interacts with the protein of interest) upon oxidation by a singlet oxygen. Upon forming the reactive intermediate, Y exhibits an increased reactivity towards a specific residue on an interactor protein or nucleic acid. Exemplary reactive groups include, but are not limited to furan, pyrrole, enol, ether, phenol, thiol and/or naphthol or derivatives thereof. Additional reactive groups include, but are not limited to, activated esters, acrylamides, acyl azides, acyl halides, acyl nitriles, aldehydes, ketones, alkyl halides, alkyl sulfonates, alkyl anhydrides, aryl halides, aziridines, boronates, carbodimides, diazoalkanes, epoxides, haloacetamides, haloplatinate, halotriazines, imido esters, isocyanates, isothiocyanates, maleimides, phosphoramidites, silyl haldies, sulfonate esters, and sulfonyl halides.

Upon reaction with a singlet oxygen, reactive groups Y generate electrophilic species that further react with a nucleophilic groups (e.g., alcohols, amines and thiols) or aromatic ring that is present in an interactor protein (e.g., cysteine, lysine, histidine, and serine side chains) or interactor nucleic acid. In some embodiments, the reactive group is a thiol or thiol derivative. In some embodiments, the thiol is an alkyl thiol, an aryl thiol, a cysteine or a peptide comprising a cysteine.

The reactive group Y can optionally be attached to the interactor detection molecule by a linker, A. In some embodiments, the interactor detection molecule includes a linker A. In other embodiments, the interactor detection molecule does not contain a linker A. Any suitable can be included in the interactor detection molecule. Suitable linkersproide optimum spacing between the reactive group and the other components of the interactor detection molecule. In certain embodiments, the linker A is an alkyl chain, an aryl, a heteroaryl, or polyethylene glycol.

In another embodiment, the interactor detection molecule has the formula:

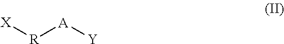

wherein X is a label, R is a cleavable peptide, A is an optional linker, Y is a reactive group capable of interaction with a cysteine, lysine histidine or serine side chain upon oxidation by a singlet oxygen (¹O₂). In such embodiments, label X, optional linker A, and reactive group Y include those label X, optional linker A, and reactive group Y described above.

Interactor detection molecules according to Formula II include a cleavable peptide R. Such cleavable peptides allow for the removal of the label X following the labeling and purification of interactor detection molecule bound interactors. Upon cleavage of the label X, the interactor can undergo subsequent characterization using any suitable method known in the art (e.g., mass spectrometry for protein interactors and nucleic acid sequencing for nucleic acid interactors). Cleavable peptides provided herein include a protease cleavage site. Any suitable protease cleavage site can be used. In certain embodiments, the cleavable peptide R contains a endoprotease cleavage site. Endoproteases include, but are not limited to, enteropeptidase, thrombin, Factor Xa, TEV protease, Rhinovirus 3C protease. In certain embodiments, the cleavable peptide contains an exoprotease cleavage site. Exoproteases include, but are not limited to, metallocarboxypeptides and aminopeptidases. Other known reagents for the removal of a label include those described in Waugh, Protein Expr Purif 80(2): 283-293 (2011), which is incorporated herein by reference.

In some embodiments, the cleavage site is a Tobacco Etch Virus (TEV) protease cleavage site. In certain embodiments, the TEV cleavage site is according to the amino acid sequence ENLYFQG (SEQ ID NO: 1) or ENLYFQS (SEQ ID NO: 2).

In an exemplary embodiment of an interactor detection molecule according to Formula II, X is biotin Y is Cysteine and the cleavage site is a TEV cleavage site.

### Interactor Detection Systems

In another aspect, provided herein is a system for the detection of interactors that interact with a protein of interest. In certain embodiments, the system includes any one of the SOG-POI fusion proteins and interactor detection molecule as described herein.

In certain embodiments, the system includes a cell expressing the SOG-POI fusion protein. Any cell type capable of expressing the SOG-POI fusion protein can be included in the system. In some embodiments, the cell is a cell in which the protein of interest is natively expressed. In certain embodiments, the cell is a cell in which the protein of interest is not natively expressed. Exemplary cells that can be used with the system, include, not are not limited to, an engineered cell; or the cell may be a recombinant cell; or the cell may be a plant cell, or the cell may be any one of an animal, a plant, an insect, an avian, yeast, algal or a fish cell. The animal cell may be mammalian, insect, bovine, primate, or a pluripotent stem cell. In one aspect, the animal cell may be a mammalian cell; and the mammalian cell may be keratinocytes, cervical epithelial cells, bronchial epithelial cells, tracheal epithelial cells, kidney epithelial cells and retinal epithelial cells) and established cell lines and their strains (e.g., 293 embryonic kidney cells, BHK cells, HeLa cervical epithelial cells and PER-C6 retinal cells, MDBK (NBL-1) cells, 911 cells, CRFK cells, MDCK cells, CHO cells, BeWo cells, Chang cells, Detroit 562 cells, HeLa 229 cells, HeLa S3 cells, Hep-2 cells, KB cells, LS180 cells, LS174T cells, NCI-H-548 cells, RPMI 2650 cells, SW-13 cells, T24 cells, WI-28 VA13, 2RA cells, WISH cells, BS-C-I cells, LLC-MK2 cells, Clone M-3 cells, 1-10 cells, RAG cells, TCMK-1 cells, Y-1 cells, LLC-PKj cells, PK(15) cells, GHj cells, GH3 cells, L2 cells, LLC-RC 256 cells, MHjCi cells, XC cells, MDOK cells, VSW cells, and TH-I, Bl cells, or derivatives thereof), fibroblast cells from any tissue or organ (including but not limited to heart, liver, kidney, colon, intestines, esophagus, stomach, neural tissue (brain, spinal cord), lung, vascular tissue (artery, vein, capillary), lymphoid tissue (lymph gland, adenoid, tonsil, bone marrow, and blood), spleen, and fibroblast and fibroblast-like cell lines (e.g., CHO cells, TRG-2 cells, IMR-33 cells, Don cells, GHK-21 cells, citrullinemia cells, Dempsey cells, Detroit 551 cells, Detroit 510 cells, Detroit 525 cells, Detroit 529 cells, Detroit 532 cells, Detroit 539 cells, Detroit 548 cells, Detroit 573 cells, HEL 299 cells, IMR-90 cells, MRC-5 cells, WI-38 cells, WI-26 cells, MiCli cells, CHO cells, CV-1 cells, COS-1 cells, COS-3 cells, COS-7 cells, Vera cells, DBS-FrhL-2 cells, BALB/3T3 cells, F9 cells, SV-T2 cells, M-MSV-BALB/3T3 cells, K-BALB cells, BLO-11 cells, NOR-10 cells, C3H/IOTI/2 cells, HSDM^{∧} cells, KLN205 cells, McCoy cells, Mouse L cells, Strain 2071 (Mouse L) cells, L-M strain (Mouse L) cells, L-MTK″ (Mouse L) cells, NCTC clones 2472 and 2555, SCC-PSA1 cells, Swiss/3T3 cells, Indian muntjac cells, SIRC cells, Cn cells, and Jensen cells, Sp2/0, NSO, NS1 cells or engineered cells thereof.

In some embodiments, the system includes an activator that is capable of activating the SOG sensitizer to generate singlet oxygen. Non-limiting examples of activators include irradiation with visible/near IR light, irradiation with ionizing radiation (i.e., x-rays or gamma rays), exposure to electromagnetic waves/materials, exposure to luminescence, exposure to fluorescence, exposure to ultrasound, and the like, as well as any combination thereof. Singlet oxygen can be generated in any methods known in the art or otherwise contemplated herein. For example, but not by way of limitation, singlet oxygen may be generated either directly from the sensitizer or indirectly by electromagnetic wave-absorbing materials coming into contact with their corresponding electromagnetic waves. When absorption of light (photon) by a sensitizer is utilized, any wavelength (UV, visible, near IR, ultrasound, radiowaves, ionizing radiation, etc.) can be used if the sensitizer observes the light and transfers the energy to oxygen (thereby yielding singlet oxygen). Optionally, light can be observed by a light harvesting material, which transfers the energy to oxygen generating singlet oxygen. Any light source known in the art or otherwise contemplated herein may be utilized; non-limiting examples include diode lasers, LED, broad band lamps with and without filters, chemo- or bio-luminescence materials, transducers (ultrasound), radioactive material or equipment (ionizing radiation), equipment for generating radio or magnetic waves, combinations thereof, and the like.

In one embodiment, the activator comprises irradiation with light in a range of from about 380 nm to about 1200 nm. Particular examples of ranges of light wavelength that may be utilized include a lower range limit of about 380 nm, about 400 nm, about 425 nm, about 450 nm, about 475 nm, about 500 nm, about 525 nm, about 550 nm, about 575 nm, about 600 nm, about 650 nm, about 675 nm, about 700 nm, about 725 nm, about 750 nm, about 775 nm, about 800 nm, about 825 nm, about 850 nm, and the like, and an upper range limit of about 1200 nm, about 1175 nm, about 1150 nm, about 1125 nm, about 1100 nm, about 1025 nm, about 1000 nm, about 975 nm, about 950 nm, about 925 nm, about 900 nm, about 875 nm, about 850 nm, about 825 nm, about 800 nm, about 775 nm, about 750 nm, and the like. In certain embodiments, short and mid-visible light, long visible light, and/or near IR light irradiation may be used as the activator.

### Interactor Detection Methods

In another aspect, provided is a method for determining interactors (e.g., proteins or nucleic acids) that interact with a protein of interesting using the systems and reagents provided herein. In certain embodiments, the method includes a step of introducing into a cell a SOG-POI fusion protein that is capable of producing singlet oxygen when illuminated by an activator and an interactor detection molecule. The SOG-POI fusion protein is then activated by an activator (e.g., a light source), thereby producing singlet oxygen molecules that converts the interactor detection molecule into a reactive intermediate. The reactive intermediate then binds nearby interactors that interact with the protein, thereby labeling in the interactors. In certain embodiments, the labeled interactors are then isolated and characterized.

Any suitable SOG-POI fusion protein described herein can be used with the subject methods. In certain embodiments, a nucleic acid encoding the SOG-POI fusion protein under the control of a promoter is introduced into the cell and the SOG-POI fusion protein is expressed from the nucleic acid. Any suitable promoter and cell expression system can be used, including those described herein.

Activators that can be used with the subject method include those described herein and depends on the SOG sensitizer used. In certain embodiments, the activator is a light source capable of illuminating a SOG photosensitizer. In some embodiments, the light source generates light that is in visible blue light (450-490 nm).

Upon activation by the activator, the SOG sensitizer converts O₂ into singlet oxygen that in turn coverts interactor detection molecules present in the cell into reactivate intermediates. Interactor detection molecules that can be used with the subject methods include those described herein. Interactor detection molecules can be introduced in the cell using any suitable method. In some embodiments, the interactor detection molecule is cell permeable. Reactive intermediates formed can then interact with specific residues on interactor proteins and nucleic acids, thereby labeling such interactors.

Cells containing the labeled interactor are lysed and the labeled interactors are separated from the cell lysate using any suitable method. As described herein, in certain embodiments, subject interactor detection molecules include a label X that facilitates purification of the interactors to which they are bound to from cell lysate (e.g., biotin, epitopes for antibodies, ligands for receptors, and nucleic acids that may interact with a second entity through means such as complementary base pair hybridization). Purified labeled interactors can be further enriched by contacting the purified labeled interactors with a protease (e.g., a serine protease, a threonine protease, a cysteine protease, an aspartate protease, a glutamic acid protease a metalloprotease). In some embodiments, the label is further removed prior to characterization of the purified interactors. In certain embodiments, the interactor detection molecule includes a cleavage site R (e.g., a TEV cleavage site) that allows removal of the label prior to characterization. In exemplary embodiments, the purified interactors undergo characterization. For example, nucleic acid interactors are identified by nucleic acid sequencing methods and protein interactors are identified by mass spectrometry.

Such methods advantageously allow for the identification of weak and transient protein-protein interactions. Such methods are useful, for example, for the determination of differences in protein-protein interactions that exist in particular diseases. This information can in turn be useful in the drug development for the treatment of such diseases.

### Preparation and Expression of Recombinant Nucleic Acids

In another aspect, provided herein are nucleic acids encoding one or more aspects of the reagents of the systems and methods provided herein. In particular embodiments, the nucleic acids encode the subject SOG-POI fusion proteins described herein. To obtain high level expression, the nucleic acid can be cloned into an expression vector that contains a strong promoter to direct transcription, a transcription/translation terminator, and if for a nucleic acid encoding a protein, a ribosome binding site for translational initiation. Suitable bacterial promoters are well known in the art and described (e.g., in Sambrook et al., and Ausubel et al., supra. Bacterial expression systems for expressing the protein are available in, e.g., E. coli, Bacillus sp., and Salmonella (Palva et al., Gene 22:229-235 (1983)); Mosbach et al., Nature 302:543-545 (1983)). Kits for such expression systems are commercially available. Eukaryotic expression systems for mammalian cells, yeast, and insect cells are well known in the art and are also commercially available. Retroviral expression systems can be used in the present invention.

Selection of the promoter used to direct expression of a heterologous nucleic acid depends on the particular application. In certain embodiments the promoter is preferably positioned about the same distance from the heterologous transcription start site as it is from the transcription start site in its natural setting. As is known in the art, however, some variation in this distance can be accommodated without loss of promoter function. Accordingly, in certain embodiments the promoter is positioned to yield optimal expression of the protein encoded by the heterologous nucleic acid. Heterologous refers to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

In addition to the promoter, in certain embodiments the expression vector also contains a transcription unit or expression cassette that contains all the additional elements required for the expression of the nucleic acid in host cells. A typical expression cassette thus contains a promoter operably linked to the nucleic acid sequence encoding the nucleic acid of choice and signals required for efficient polyadenylation of the transcript, ribosome binding sites, and translation termination. In certain embodiments, additional elements of the cassette can include enhancers and, if genomic DNA is used as the structural gene, introns with functional splice donor and acceptor sites.

In addition to a promoter sequence, the expression cassette should also contain a transcription termination region downstream of the structural gene to provide for efficient termination. The termination region can be obtained from the same gene as the promoter sequence or can be obtained from different genes.

One of skill in the art will know how to select an expression vector based on the size of the insert and the cell-type to be transfected or transformed. For example, any of the conventional vectors used for expression in eukaryotic or prokaryotic cells can be used. Standard bacterial expression vectors include plasmids such as pBR322 based plasmids, pSKF, pET23D, and fusion expression systems such as MBP, GST, and LacZ. Epitope tags can also be added to recombinant proteins to provide convenient methods of isolation, e.g., c-myc. Sequence tags can be included in an expression cassette for nucleic acid rescue. Markers such as fluorescent proteins, green or red fluorescent protein, 13-gal, CAT, and the like can be included in the vectors as markers for vector transduction.

In certain embodiments, regulatory elements can be incorporated into the expression vectors. Expression vectors containing regulatory elements include but are not limited to SV40 vectors, papilloma virus vectors, retroviral vectors, and vectors derived from Epstein-Barr virus. Other exemplary eukaryotic vectors include but are not limited to pMSG, pAV009/A⁺, pMT010/A⁺, pMAMneo-5, baculovirus pDSVE, and any other vector allowing expression of proteins under the direction of the CMV promoter, SV40 early promoter, SV40 later promoter, metallothionein promoter, murine mammary tumor virus promoter, Rous sarcoma virus promoter, polyhedrin promoter, or other promoters shown effective for expression in eukaryotic cells.

In certain embodiments expression of proteins from eukaryotic vectors can also be regulated using inducible promoters. With inducible promoters, expression levels are tied to the concentration of inducing agents, such as tetracycline, by the incorporation of response elements for these agents into the promoter. Generally, high level expression is obtained from inducible promoters only in the presence of the inducing agent; basal expression levels are minimal.

In certain embodiments, a multicistronic vector comprises a nucleic acid that encodes an IFP disclosed herein and one or more additional genes.

In certain embodiments the vector includes a regulatable promoter, e.g., tet-regulated systems and the RU-486 system (see, e.g., Gossen & Bujard, PNAS 89:5547 (1992); Oligino et al., Gene Ther. 5:491-496 (1998); Wang et al., Gene Ther. 4:432-441 (1997); Neering et al., Blood 88:1147-1155 (1996); and Rendahl et al., Nat. Biotechnol. 16:757-761 (1998)). These impart small molecule control on the expression of the candidate target nucleic acids. This beneficial feature can be used to determine that a desired phenotype is caused by a transfected cDNA rather than a somatic mutation.

Some expression systems have markers that provide gene amplification such as thymidine kinase and dihydrofolate reductase. Alternatively, high yield expression systems not involving gene amplification are also suitable, such as using a baculovirus vector in insect cells, with a sequence of choice under the direction of the polyhedrin promoter or other strong baculovirus promoters.

Additional elements that are incorporated into expression vectors include but are not limited to a replicon that functions in E. coli, a gene encoding antibiotic resistance to permit selection of bacteria that harbor recombinant plasmids, and unique restriction sites in nonessential regions of the plasmid to allow insertion of eukaryotic sequences. The particular antibiotic resistance gene chosen is based on various factors. For example, antibiotic resistance genes will be chosen and incorporated into an expression vector based on the organism and/or cell line that is to be transfected/transformed. In other examples, antibiotic resistance genes are chosen based on the a series of co-transfections and multi-gene selection criteria. The prokaryotic sequences are preferably chosen such that they do not interfere with the replication of the DNA in eukaryotic cells, if necessary.

Standard transfection and transformation techniques are known to one of skill in the art. These techniques can be used to produce bacterial, mammalian, yeast or insect cell lines that express large quantities of protein, which are then purified using standard techniques (see, e.g., Colley et al., J. Biol. Chem. 264:17619-17622 (1989); Guide to Protein Purification, in Methods in Enzymology, vol. 182 (Deutscher, ed., 1990)). In certain embodiments, transformation and transfection of eukaryotic and prokaryotic cells are performed according to standard techniques (see, e.g., Morrison, J. Bact. 132:349-351 (1977); Clark-Curtiss & Curtiss, Methods in Enzymology 101:347-362 (1983)).

Furthermore, one of skill in the art will know that any of the well-known procedures for introducing foreign nucleotide sequences into host cells (e.g., transformation or transfection) can be used. These include the use of calcium phosphate transfection, polybrene, protoplast fusion, electroporation, biolistics, liposomes, microinjection, plasma vectors, viral vectors and any of the other well known methods for introducing cloned genomic DNA, cDNA, synthetic DNA or other foreign genetic material into a host cell (see, e.g., Sambrook et al., supra). It is only necessary that the particular genetic engineering procedure used be capable of successfully introducing at least one gene into the host cell capable of expressing mIFP proteins and nucleic acids.

After the expression vector is introduced into the cells, the transfected cells are cultured under conditions favoring expression of the protein of choice, which is recovered from the culture using standard techniques identified below.

Recombinant SOG-POI fusion proteins can be purified, for example, for use in protein-protein interaction assays as described herein. Recombinant protein can be purified from any suitable expression system.

### Purification of SOG-POI Fusion Proteins

The SOG-POI fusion proteins described herein can be purified to substantial purity by standard techniques, including selective precipitation (e.g., with such substances such as ammonium sulfate), column chromatography, immunopurification methods, and other techniques known to one of skill in the art (see, e.g., Scopes, Protein Purification: Principles and Practice (1982); U.S. Pat. No. 4,673,641; Ausubel et al., supra; and Sambrook et al., supra).

A number of procedures known to one of skill in the art can be employed when recombinant protein is being purified. For example, proteins having established molecular adhesion properties can be reversible fused to the protein. With the appropriate ligand or substrate, a specific protein can be selectively adsorbed to a purification column and then freed from the column in a relatively pure form. The fused protein is then removed by enzymatic activity. Finally, protein can be purified using immunoaffinity columns. Recombinant protein can be purified from any suitable source, include yeast, insect, bacterial, and mammalian cells.

Recombinant proteins can be expressed and purified by transformed bacteria in large amounts, typically after promoter induction; but expression can be constitutive. For example, promoter induction with IPTG is one example of an inducible promoter system. Bacteria are grown according to standard procedures in the art. Fresh or frozen bacteria cells are used for isolation of protein.

Proteins expressed in bacteria can form insoluble aggregates (“inclusion bodies”). Several protocols are suitable for purification of protein inclusion bodies. For example, purification of inclusion bodies typically involves the extraction, separation and/or purification of inclusion bodies by disruption of bacterial cells, e.g., by incubation in a buffer of 50 mM TRIS/HCl pH 7.5, 50 mM NaCl, 5 mM MgCl2, 1 mM DTT, 0.1 mM ATP, and 1 mM PMSF. One of skill in the art will know that the stability of the protein will determine the lysis and purification buffer components. For example, one of skill in the art will know that protease inhibitor cocktails can be used to minimize and/or prevent protein degradation. Such protease inhibitor cocktails may include but are not limited to PMSF. Similarly, one of skill in the art will know that detergents and/or surfactants may be added to prevent protein aggregation, to enhance purification, and increase solubilization. Specifically, non-ionic, zwitterionic, and ionic detergents can be used. Such detergents and surfactants include but are not limited to Tween, Triton (e.g., Triton X-100), octylglucoside, DM, DDM, Chaps, Zwittergents (e.g., zwittergent 3-12), sodium deoxycholate, and glycerol. Furthermore, one of skill in the art will know that reducing agents can be used to prevent aggregation and enhance purification of proteins. Reducing agents include, but are not limited to, 2-mercaptoethanol, DTT, and TCEP. The cell suspension can be lysed using 2-3 passages through a French Press, homogenized using a Polytron (Brinkman Instruments) or sonicated on ice. Alternate methods of lysing bacteria are apparent to those of skill in the art (see, e.g., Sambrook et al., supra; Ausubel et al., supra).

If necessary, the inclusion bodies are solubilized, and the lysed cell suspension is typically centrifuged to remove unwanted insoluble matter. Proteins that formed the inclusion bodies can be renatured by dilution or dialysis with a compatible buffer. Suitable solvents include, but are not limited to urea (from about 4 M to about 8 M), formamide (at least about 80%, volume/volume basis), and guanidine hydrochloride (from about 4 M to about 8 M). Some solvents which are capable of solubilizing aggregate-forming proteins, for example SDS (sodium dodecyl sulfate), 70% formic acid, are inappropriate for use in this procedure due to the possibility of irreversible denaturation of the proteins, accompanied by a lack of immunogenicity and/or activity. Although guanidine hydrochloride and similar agents are denaturants, this denaturation is not irreversible and renaturation can occur upon removal (by dialysis, for example) or dilution of the denaturant, allowing re-formation of immunologically and/or biologically active protein. Other suitable buffers are known to those skilled in the art. Human proteins are separated from other bacterial proteins by standard separation techniques, e.g., with Ni-NTA agarose resin.

Alternatively, it is possible to purify recombinant protein from bacteria periplasm. After lysis of the bacteria, the periplasmic fraction of the bacteria can be isolated by cold osmotic shock in addition to other methods known to skill in the art. To isolate recombinant proteins from the periplasm, the bacterial cells are centrifuged to form a pellet. The pellet is resuspended in a buffer containing 20% sucrose. To lyse the cells, the bacteria are centrifuged and the pellet is resuspended in ice-cold 5 mM MgSO₄ and kept in an ice bath for approximately 10 minutes. The cell suspension is centrifuged and the supernatant decanted and saved. The recombinant proteins present in the supernatant can be separated from the host proteins by standard separation techniques well known to those of skill in the art.

Solubility fractionation can be used as a standard protein separation technique for purifying proteins. As an initial step, particularly if the protein mixture is complex, an initial salt fractionation can separate many of the unwanted host cell proteins (or proteins derived from the cell culture media) from the recombinant protein of interest. The preferred salt is ammonium sulfate. Ammonium sulfate precipitates proteins by effectively reducing the amount of water in the protein mixture. Proteins then precipitate on the basis of their solubility. The more hydrophobic a protein is, the more likely it is to precipitate at lower ammonium sulfate concentrations. A typical protocol includes adding saturated ammonium sulfate to a protein solution so that the resultant ammonium sulfate concentration is between 20-30%. This concentration will precipitate the most hydrophobic of proteins. The precipitate is then discarded (unless the protein of interest is hydrophobic) and ammonium sulfate is added to the supernatant to a concentration known to precipitate the protein of interest. The precipitate is then solubilized in buffer and the excess salt removed if necessary, either through dialysis or diafiltration. Other methods that rely on solubility of proteins, such as cold ethanol precipitation, are well known to those of skill in the art and can be used to fractionate complex protein mixtures.

The molecular weight of the protein can be used to isolate it from proteins of greater and lesser size using ultrafiltration through membranes of different pore size (for example, Amicon or Millipore membranes). As a first step, the protein mixture is ultrafiltered through a membrane with a pore size that has a lower molecular weight cut-off than the molecular weight of the protein of interest. The retentate of the ultrafiltration is then ultrafiltered against a membrane with a molecular cut off greater than the molecular weight of the protein of interest. The recombinant protein will pass through the membrane into the filtrate. The filtrate can then be chromatographed as described below.

The protein can also be separated from other proteins on the basis of its size, net surface charge, hydrophobicity, and affinity for ligands or substrates using column chromatography. In addition, antibodies raised against proteins can be conjugated to column matrices and the proteins immunopurified. All of these methods are well known in the art. It will be apparent to one of skill that chromatographic techniques can be performed at any scale and using equipment from many different manufacturers (e.g., Pharmacia Biotech).

The invention provides kits for practicing the assays described herein. Kits for carrying out the diagnostic assays of the invention typically include a probe that comprises an antibody or nucleic acid sequence that specifically binds to polypeptides or polynucleotides of the invention, and a label for detecting the presence of the probe. The kits may include several antibodies or polynucleotide sequences encoding polypeptides of the invention, e.g., a cocktail of antibodies that recognize the proteins encoded by the biomarkers of the invention.

### EXAMPLES

The methods system herein described are further illustrated in the following examples, which are provided by way of illustration and are not intended to be limiting.

### Example 1: System for Labeling Skp2 Interactors

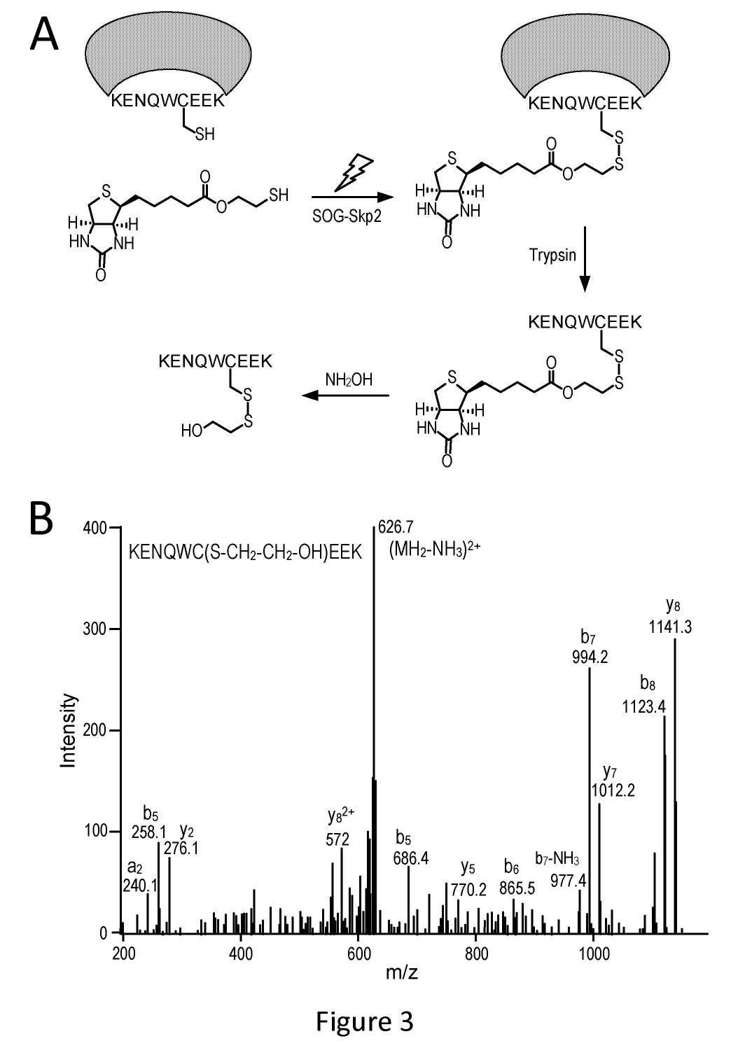

As a proof of concept of the system, reagents and methods described herein, a biotin-conjugated thiol-containing molecule: 2-mercaptoethyl 5-((3aS,4S,6aR)-2-oxohexahydro-1H-thieno[3,4-d]imidazol-4 yl)pentanoate, with chemical formula: C₁₂H₂₀N₂O₃S₂ (FIG. 3) was designed. Based on this molecule, the subject systems and methods provided herein are demonstrated in chemical labeling of interactors for an exemplary protein of interest (Skp2). The experiments were carried out with the following procedures (FIG. 3):

A singlet oxygen photosynthesizer (SOG) was fused to Skp2, an F-box protein of SCF (Skp1-cullin-F-Box) ubiquitin ligase to create a SOG-Skp2 fusion protein. SCF is a multi-protein complex, composed of four subunits: Skp1 (S-phase kinase-associated protein 1), cullin1, a RING finger protein (RBX1/HRT1/ROC1) and a variable F-box protein.

The SOG-skp2 fusion protein was expressed in cultured mammalian cells, HeLa.

The designed labeling molecules were added to the cells, followed by illumination with 480/30 nm light for 10-30 minutes.

The cells were lysed, followed by centrifugation. The labeled interactors were incubated with trypsin and loaded to streptavidin beads, and separated from the rest by streptavidin-biotin interaction.

The labeled peptides were eluted by incubation with amine hydroxyl, which cleaved the labeled peptides from streptavidin beads.

These interaction peptides were identified by mass spectrometry.

Based on mass spectrometry, skp1, which interacts with skp2, was labeled and identified by the subject systems and methods. Specifically, protein interaction detection molecule specifically labeled the cysteine of the unique peptide from skp1, KENQWCEEK (FIG. 3).

### Example 2: System for Labeling Phycobiliprotein, Sllophycocyanin (APC) Interactors

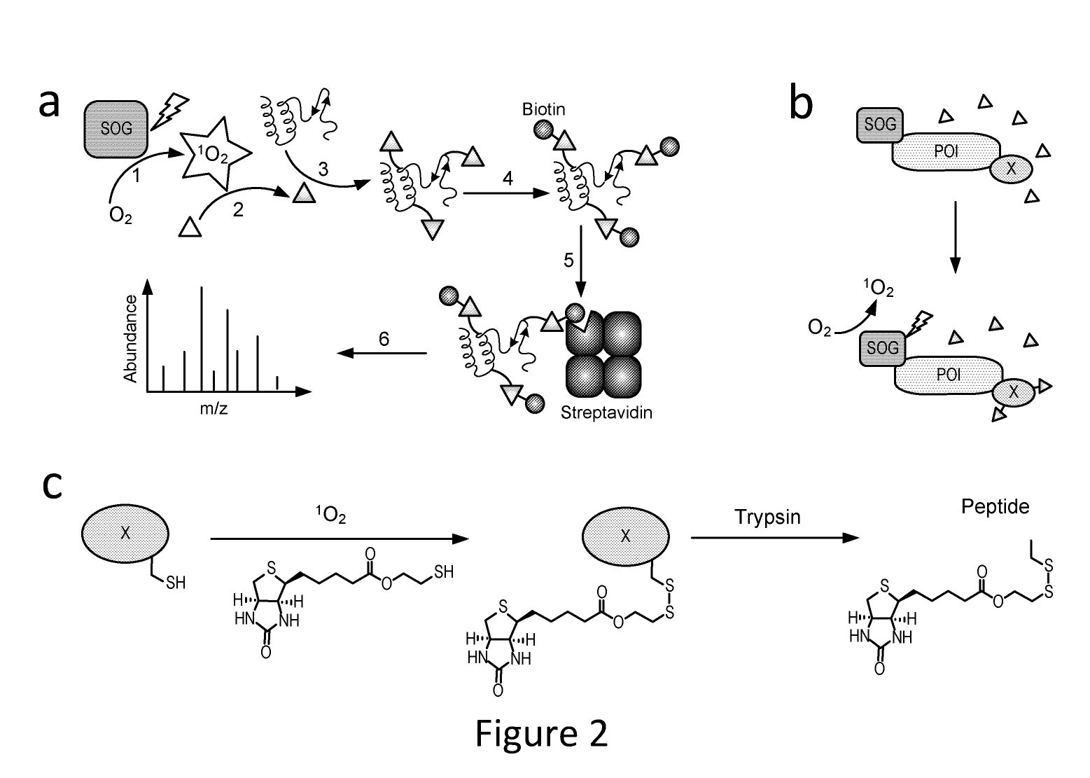

To realize the concept of the subject systems, reagents and methods provided herein, a genetically encoded small ¹O₂ photosensitizer miniSOG (106-residue) that requires no exogenous cofactors (Shu et al., PLoS Biology 9: e1001041 (2011)) was used. MiniSOG is green fluorescent and generates ¹O₂ when illuminated with blue light (450-490 nm). Secondly, a labeling molecule was designed to be small and cell permeable, and most importantly, upon singlet oxygen activation, it has strong reactivity toward a specific protein residue. One class of such chemicals is thiol-based molecule. Thus, a small molecule containing a thiol group attached to biotin (FIG. 2C) toward a specific protein residue was specifically designed.

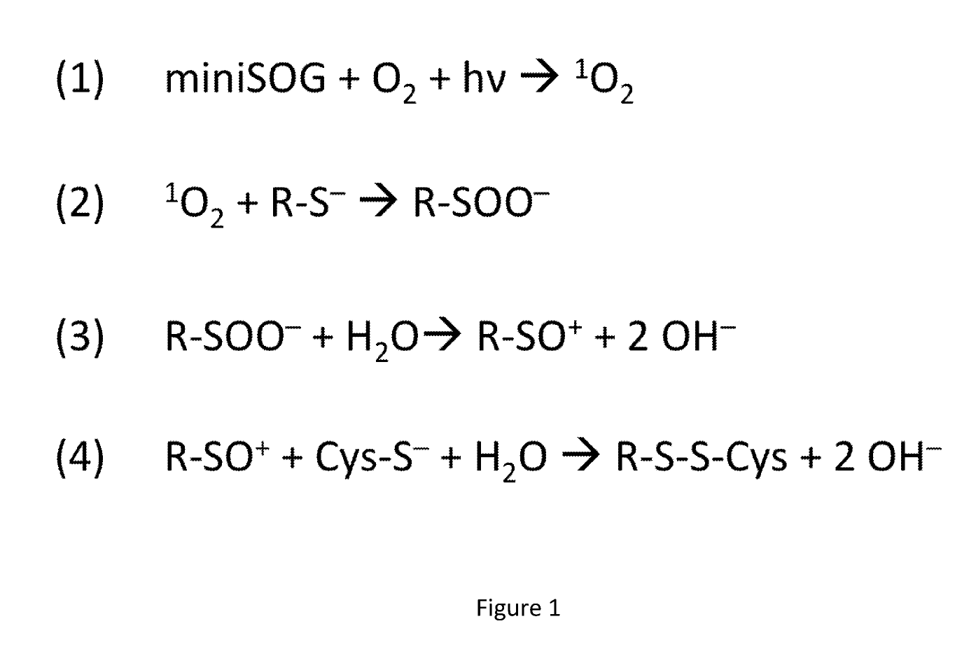

Here light was used to generate ¹O₂ from miniSOG. ¹O₂ then oxidizes the thiol group, which reacts with cysteine on protein surfaces, forming a disulfide bond (FIG. 1)(Buettner et al., Biochim Biophys Acta 923: 501-507 (1987)). The labeled proteins can then be separated from the rest through streptavidin-biotin interaction. The labeled peptides can be further enriched by trypsinizing the separated proteins (FIG. 2C). These biotin labeled peptides can be eluted by biotin competition, for example, by desthiobiotin. Lastly, these eluted peptides can be sequenced and identified by tandem mass spectrometry.

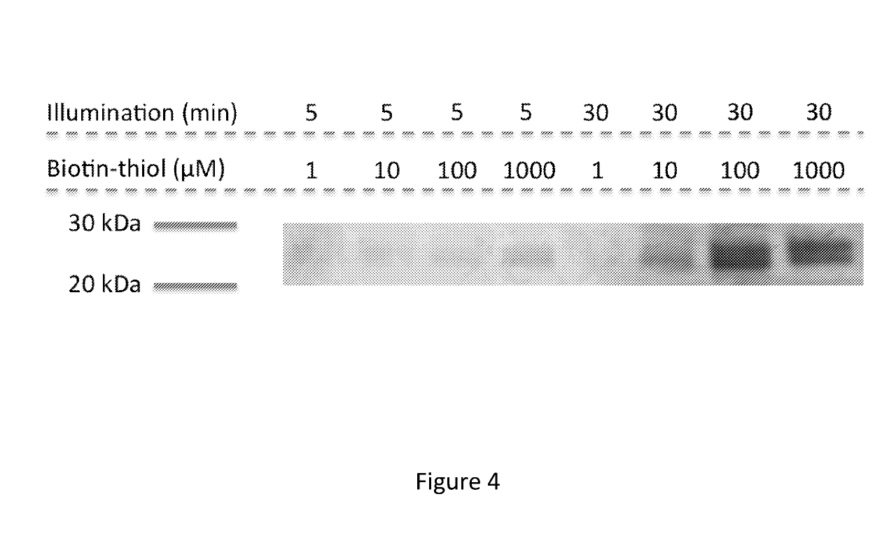

As a proof of concept, miniSOG was directly fused to a phycobiliprotein, allophycocyanin (APC) that contains three cysteine residues, and tested the labeling method. Freshly purified miniSOG-APC protein (1 μM) was mixed with various concentrations of the biotin-thiol molecule, and labeling experiments were performed with different illumination times. The reaction mixture was illuminated with a customized 450±20 nm blue LED (Innovations in Optics, Woburn Mass.) at an intensity of ˜200 mW/cm². After illumination, we the free labeling molecules were first removed by centrifugal filtration. Then the labeled proteins by streptavidin, and assayed the labeled proteins by LDS-PAGE (FIG. 4). The data indicated that the amount of labeled proteins depends on both the illumination time and the concentration of labeling molecules. For example, with 10 μM biotin-thiol, the longer the illumination time was, the higher the labeling efficiency. Efficient labeling was achieved with 100 μM biotin-thiol and 30-minute illumination time. Under such conditions, about 75% of the fusion protein was recovered. On the other hand, when the illumination time was reduced to 5 minutes, negligible amount of proteins were labeled with 100 μM biotin-thiol.

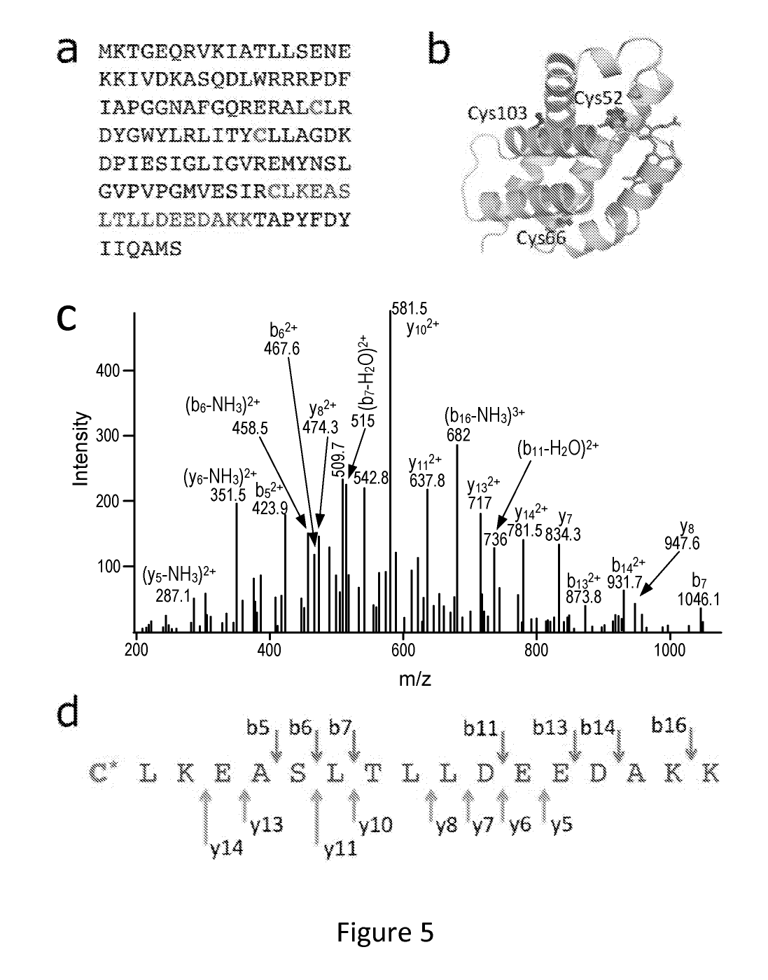

To confirm the specific chemical modification, we trypsinized the streptavidin-bound proteins, which were then eluted and subjected to LC/MS/MS experiment using an LTQOrbitrap XL mass spectrometer. While miniSOG does not contain cysteine, APC contains three cysteine residues according to the primary sequence (FIG. 5A), including Cys52, Cys66 and Cys103. Furthermore, based on the modeled structure (Swiss-Model) (Schwede, Nucleic Acids Res 31: 3381-3385 (2003)), Cys103 resides on the protein surface (FIG. 5B), whereas Cys52 and Cys66 are buried inside the protein, with the former linked to the cofactor phycocyanobilin (in purple). Therefore, we expected that the exposed cysteine Cys103 would be labeled by the biotin-thiol molecule. Indeed, LC/MS/MS identified the Cys103-bearing tryptic peptide CLKEASLTLLDEEDAKK (FIG. 5A, C), with an ion trap CID (collision induced dissociation) of m/z 552.5249(4+). The N-terminal Cys103 was identified as carrying the biotin-thiol signature (FIG. 5C). Various fragment ions derived from the peptide were detected (FIG. 5C). The nomenclature of these ions (FIG. 5C) is that (FIG. 5D): the green arrows indicate cleavage sites where fragments with N-terminal charge retention were formed (b or related ions); the red arrows indicate cleavage sites when C-terminal sequence ions (y or related fragments) were formed. The LC/MS/MS results thus confirmed that Cys103 was correctly labeled by the biotin-thiol molecule as expected. On the other hand, the mass spectrometry results suggested that the buried cysteines Cys52 and Cys66 were not labeled, presumably because they were not accessible to the labeling molecules.

Thus, a singlet oxygen mediated, photo-activatable protein labeling technology was developed. This system has applications in molecular and cell biology as well as in disease. For example, since ¹O₂ has a short diffusion distance, interacting proteins that are proximal to the protein of interest (fused to miniSOG) can be specifically labeled and identified by mass spectrometry. Such applications include identification of E3 ligase substrates (Shi et al., Mol Cell Proteomics 10: R110.006882 (2011)), deorphanization of G-protein coupled receptors (Lagerström and Schiöth Nat Rev Drug Discov 7: 339-357 (2008); and Bjarnadóttir et al., Cell Mol Life Sci 64: 2104-2119 (2007)). Such an in situ protein labeling method enables for the map protein interactome in the native cellular context of cultured cells and animal tissues. Further, it allows for the investigation of perturbation of protein interactome in the diseased human tissues (Vidal et al., Cell 144: 986-998 (2011); and Ideker, T., and Sharan, R. Genome Research 18: 644-652 (2008)), which will not only help our understanding of molecular mechanisms of disease but also identify new targets for therapeutic intervention.

Methods

Gene Construction and Protein Purification

MiniSOG-APC was expressed with C-terminal polyhistidine-tag on a pBAD expression vector (Invitrogen). A 5 aa SGGGS linker was inserted between miniSOG and APC. For phycocyanobilin and biliverdin production in E. coli, the PCB:ferredoxin oxidoreductase (PcyA) and heme oxygenase-1 (HO1) genes codon-optimized for E. coli were coexpressed on the same vector. The resulted plasmid construct was created by standard molecular biology techniques and confirmed by sequencing the cloned fragments. The fusion protein was purified with the Ni-NTA purification system (Qiagen). The protein concentration was quantified based on the absorbance of miniSOG and its published extinction coefficient.

Synthesis and Purification of the Biotin-Thiol Labeling Molecule

A biotin-conjugated thiol-containing molecule was synthesized by esterification reaction using a slight modification of the literature method (Wulff et al., J Am Chem Soc 129:4898-4899 (2007)) followed by reduction with TCEP. Briefly, 2-hydroxyethyldisulfide (476 mg, 3 mmol) and triethylamine (5 mL) were added to a solution of (+)-biotin (1.00 g, 4 mmol), N-(3-dimethylaminopropyl)-N′-ethylcarbodiimide hydrochloride (EDC.HCl, 942 mg, 4.9 mmol), and 1-hydroxybenzotriazole (HOBt, 664 mg, 4.9 mmol) in N,N-dimethylformamide (DMF, 15 mL). The reaction mixture was stirred at room temperature overnight and diluted with dichloromethane. The reaction mixture was washed with 1M hydrochloric acid solution and the aqueous layer was extracted with dichloromethane twice. The combine organic layer was washed with brine, dried over sodium sulfate and concentrated. The crude mixture was filtered through a short pad of silica (chloroform:methanol 9:1). A mixture of the above disulfide (200 mg), Tris(2-carboxyethyl)phosphine hydrochloride (TCEP.HCl, 174 mg) in 30% acetonitrile in H₂O (6 mL) was stirred for 2 h at room temperature and lyophilized. The solid was purified by RP-HPLC (Atlantis T3 OBD column, 19.5×150 mm, solvent A: 0.1% TFA in H2O, solvent B: 0.1% TFA in i-PrOH:CH3CN:H2O (6:3:1), gradient: 5% solvent B to 100% solvent B over 31 min after 5 min 5% solvent B, flow rate 15 ml/min). The biotin-ester thiol has retention time 18.8 min, 1H NMR (CDCl3, 300 MHz) δ ppm 5.59 (s, 1H), 5.16 (s, 1H), 4.49 (s, 1H), 4.32 (s, 1H), 4.21-4.17 (m, 2H), 3.15 (s, 1H), 2.94-2.81 (m, 1H), 2.78-2.71 (m, 3H), 2.39-2.34 (m, 2H), 1.72-1.47 (m, 6H) ESI-MS: calcd. m/z=305.1 (MH+), found 305.6.

Photo-Activatable Protein Labeling

Various concentrations of the biotin-thiol substrate were added to 1 μM of miniSOGAPC in a reaction volume of 100 μL in phosphate-buffered saline (PBS) buffer at pH 7.4. To photo-generate 1O2, the reaction mixture was illuminated with a 450±20 nm blue LED (Innovations in Optics, Woburn Mass.) at an intensity of ˜200 mW/cm2 at room temperature. After the reaction, the mixture was purified with an Amicon centrifugal filter of 3-kDa cutoff (EMD Millipore), in order to remove the free biotin-thiol molecules. The streptavidin-biotin purification of labeled protein was performed with 100 μL of Strep-Tactin Superflow resin (IBA). Elution was done by 10 mM of desthiobiotin (Sigma-Aldrich). The purified labeled protein was then assayed by an LDS-PAGE using NuPAGE® Novex® 4-12% Bis-Tris protein gel (Life Technologies). The protein gel was imaged with white light in an AlphaImager 3300 Imaging System (Alpha Innotech).

Tandem Mass Spectrometry

In-solution trypsinization of the purified labeled protein was done in 50 mM ammonium bicarbonate (pH 8). Trypsin (Promega) was added to the protein at 1:50 trypsin:protein ratio (w/w). The mixture was incubated at 37° C. for 3 hours and desalted with a SepPak C18 cartridge (Waters Corp.). After desalting, the sample was dried with a centrifugal evaporator (GeneVac) and stored at −80° C. The dried sample was resuspended in 100 μL of 0.1% formic acid for LC/MS/MS analysis. Mass spectrum data was acquired in a data-dependent LC/MS/MS experiment using an LTQ-Orbitrap XL mass spectrometer. The precursor ion was measured in the Orbitrap, and is within 1 ppm of the calculated value. Fragment ions detected are labeled according to the nomenclature (Biemann, Meth Enzymol 193: 886 (1990)).

1. An interactor detection molecule according to the formula: 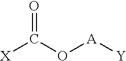
wherein:
X is a label;
A is an optional linker; and
Y is a reactive group capable of interaction with a cysteine, lysine histidine or serine side chain upon oxidation by a singlet oxygen (¹O₂).

2. The interactor detection molecule of claim 1, wherein the label X is a radioisotope, a stable isotope, a fluorophore, an electron dense metal, biotin, a nucleic acid or an antibody epitope.

3. The interactor detection molecule of claim 1, wherein the linker A is an alkyl chain, an aryl, a heteroaryl, or polyethylene glycol.

4. The interactor detection molecule of claim 1, wherein the reactive group Y is a thiol, a furan, a pyrrole, an enol ether, a phenol or a naphthol or derivative.

5. The interactor detection molecule of claim 4, wherein the thiol is selected from an alkyl thiol, an aryl thiol, a cysteine and a peptide comprising a cysteine.

6. The interactor detection molecule of claim 1, wherein X is biotin, A is CH₂CH₂, and Y is a thiol containing group.

7. An interactor detection molecule having a formula: 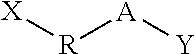
wherein:
X is a label;
R is a cleavable peptide;
A is an optional linker;
Y is a reactive group capable of interaction with a cysteine, lysine histidine or serine side chain upon oxidation by a singlet oxygen (¹O₂).

8. The interactor detection molecule of claim 7, wherein the label X is a radioisotope, a stable isotope, a fluorophore, an electron dense metal, biotin, a nucleic acid or an antibody epitope.

9. The interactor detection molecule of claim 7, wherein the linker A is an alkyl chain, an aryl, a heteroaryl, or polyethyleneglycol.

10. The interactor detection molecule of claim 7, wherein the cleavable peptide R comprises a protease cleavage site.

11. The interactor detection molecule of claim 10, wherein the protease cleavage site is a Tobacco Etch Virus protease cleavage site.

12. The interactor detection molecule of claim 7, wherein the linker A is an alkyl chain, an aryl, a heteroaryl, or polyethylene glycol.

13. The interactor detection molecule of claim 7, wherein the reactive group is a thiol, a furan, a pyrrole, an enol ether, a phenol or a naphthol or derivative.

14. The interactor detection molecule of claim 13, wherein the thiol is selected from an alkyl thiol, an aryl thiol, a cysteine and a peptide comprising a cysteine.

15. The interactor detection molecule of claim 1, wherein X is biotin and Y is a cysteine.

16. A system for determining interactors that interact with a protein of interest comprising:
a SOG-POI protein comprising a singlet oxygen photosensitizer fused to a protein of interest, wherein the singlet oxygen photosensitizer is capable of producing singlet oxygen (¹O₂) when illuminated with a light source; and
the interactor detection molecules of claim 1.

17. A cell comprising:
a SOG-POI protein comprising a singlet oxygen photosensitizer fused to a protein of interest, wherein the singlet oxygen photosensitizer is capable of producing singlet oxygen (¹O₂) when illuminated with a light source; and
the interactor detection molecules of claim 1.

18. A method for determining interactors that interact with a protein of interest comprising:
a) introducing into a cell a SOG-POI protein and an interactor detection molecule selected from the interactor detection molecules of claim 1, wherein the SOG-POI protein comprises a singlet oxygen photosensitizer fused to a protein of interest, and wherein the singlet oxygen photosensitizer is capable of producing singlet oxygen (¹O₂) when illuminated with a light source;
b) illuminating the singlet oxygen photosensitizer with a light source, thereby producing singlet oxygen (¹O₂) that oxidizes the interactor detection molecule to form a reactive detection intermediate, wherein the reactive detection intermediate binds with a interaction protein that interacts with the protein of interest, thereby labeling the interactor; and
c) characterizing the interactor.

19. The method of claim 18, wherein the c) characterizing is carried out by isolating the interactive intermediate bound interactors and the interactor is sequenced by mass spectrometry.

20. The method of claim 19, wherein X of the interactor detection molecule is a biotin and isolation of the interactive protein is performed using a substrate attached to streptavidin.
